(19) United States
(12) Patent Application Publication
(10) Pub. No.: US 2013/0023843 A1
(43) Pub. Date: Jan. 24, 2013

(54) Urinary Leg Bag Holder and Straps

(71) Applicant: Dan Chen, Guangzhou (CN)

(72) Inventor: Dan Chen, Guangzhou (CN)

(21) Appl. No.: 13/185,470

(22) Filed: Jul. 18, 2011

(51) Int. Cl. A61F 5/44

(52) U.S. Cl. 604/327

(57) ABSTRACT

This invention provides a urinary drainage leg bag supporter comprising a waist belt; an adjustable enclosure on the belt; a pouch suspending from the enclosure for holding the urinary drainage leg bag, the pouch is formed by folding up one piece of octagonal fabric, connected at the boarders by eight sets of snap fasteners; two sets of buttons and one set of snap fasteners inside of the pouch firmly holds the urinary drainage leg bag in place; two sets of adjustable, stretchable horizontal straps extending from the sides of the pouch to stabilize the pouch on the wearer's thigh.

### BACKGROUND OF THE INVENTION

1. Field of the Invention

This invention relates to urinary drainage leg bag supporters, specifically, belt and straps supported urinary leg drainage bag supporters.

2. Description of the Prior Art

Patients with urological diseases who had lost their abilities to urinate naturally are usually provided with a device for collecting and storing urine. A common method comprises fitting the patient with an indwelling drainage tube with extension that drains urine directly into a vinyl or plastic leg bag with an inlet in the top portion of the bag and a drain valve in the bottom. This plastic leg bag is usually fastened onto the patient's thigh with two sets of straps. This method of urine collection device is oftentimes warm and uncomfortable as the vinyl or plastic material of the leg bag tends to stick and rub against the skin. As the bag becomes filled with urine, the straps are incapable of holding up the bag tight against the patient's thigh, causing a downward pull to the indwelling drainage tube which can cause the patient great discomfort or pain. More importantly, the appearance of the leg bag can cause great embarrassment and distress to the patient in many social scenes.

From the above description, the current method of securing drainage leg bag is highly uncomfortable and inconvenient to the patient, and can potentially cause great embarrassment. The present invention is made to correct the aforementioned problems.

### SUMMARY OF INVENTION

This invention provides a urinary drainage leg bag supporter comprising a waist belt, an adjustable enclosure on the belt with a U shape fabric woven tape; a pouch suspending from the enclosure by the means of snap fasteners on the U shape tape of the aforementioned enclosure for holding the urinary drainage leg bag, the pouch is formed by folding up one piece of octagonal fabric, connected at the boarders by eight sets of snap fasteners; two sets of buttons and one set of snap fasteners inside of the pouch firmly holds the urinary drainage leg bag in place; two sets of adjustable, stretchable horizontal straps extending from the sides of the pouch to stabilize the pouch on the wearer's thigh.

This invention is adjustable in accordance to the drainage location and the measurement of the patient. Most patients can put it on by themselves, as well as replacing the drainage leg bag with ease. The Urinary Leg Bag Holder and Straps is worn between patient's underwear and pants/skirt, provides convenience to the patient who need to use the toilet normally. This invention is suitable for most vinyl and plastic urinary leg bags. It offers its user security, comfort and convenience over the standard leg straps packaged with most leg bags, and reduces the chance of leg bag rupture due to friction. This invention is reusable, durable and washable.

### BRIEF DESCRIPTION OF THE DRAWINGS

FIG. 1 shows the present Urinary Leg Bag Holder and Straps as worn by the patient, with indwelling tube on different location of the patient, viewing from front, back and side.

FIG. 2 illustrates the details of the belt of the present Urinary Leg Bag Holder and Straps.

FIG. 3 illustrates the details of the adjustable enclosure that connects the belt and the pouch.

FIG. 4 illustrates the detail exterior view of the pouch of the present Urinary Leg Bag Holder and Straps.

FIG. 5 illustrates the detail interior view of the pouch of the present Urinary Leg Bag Holder and Straps.

FIG. 6 illustrates a three dimensional view of the present Urinary Leg Bag Holder and Straps after all the parts are properly assembled.

FIG. 7 shows a three dimensional view of the present Urinary Leg Bag Holder and Straps at its alternative position adjustable by wearer.

FIG. 8 shows the present Urinary Leg Bag Holder and Straps with conventional leg bag attached.

FIG. 9 shows the side of the present Urinary Leg Bag Holder and Straps adjacent to the wearer's body.

### DETAILED DESCRIPTION

This invention provides support to conventional urinary drainage leg bag, binds and contains the leg bag irrespective of the position of the wearer.

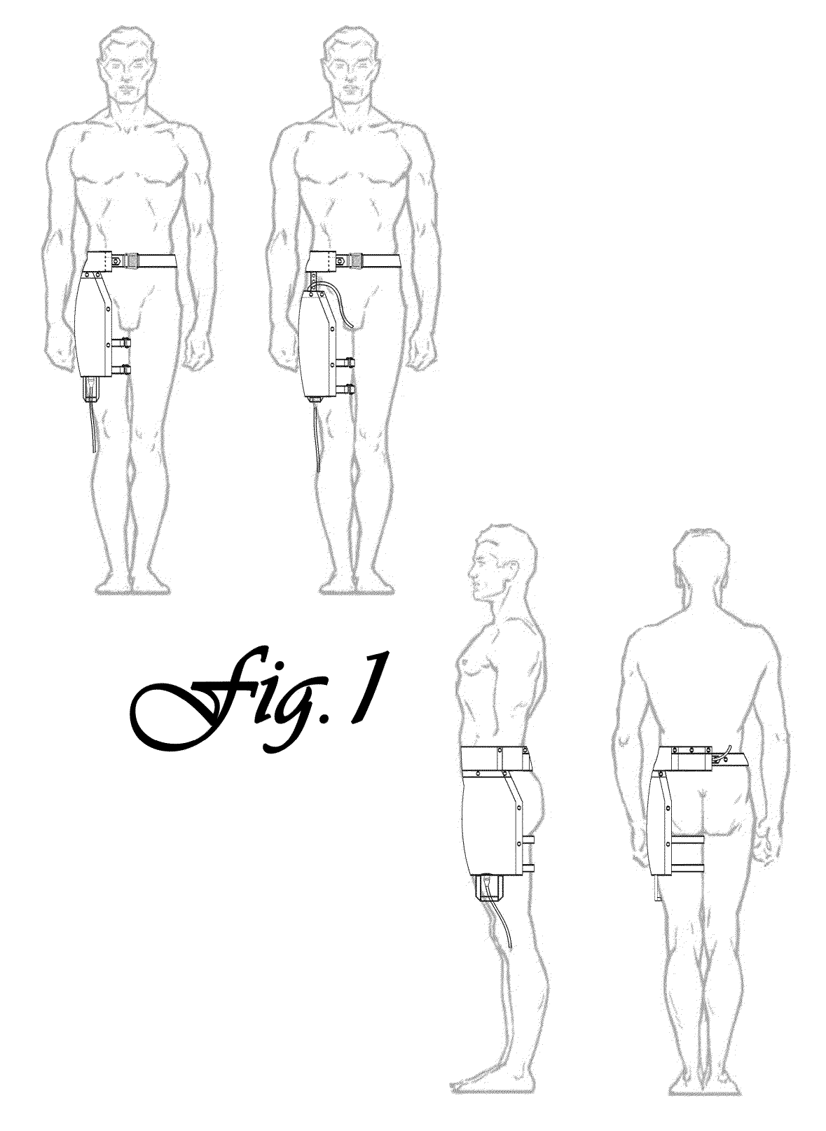

With reference to the drawings, FIG. 1 shows the invention being worn by a patient. Further details of the structure and composition of the invention are shown in FIGS. 2 to 5.

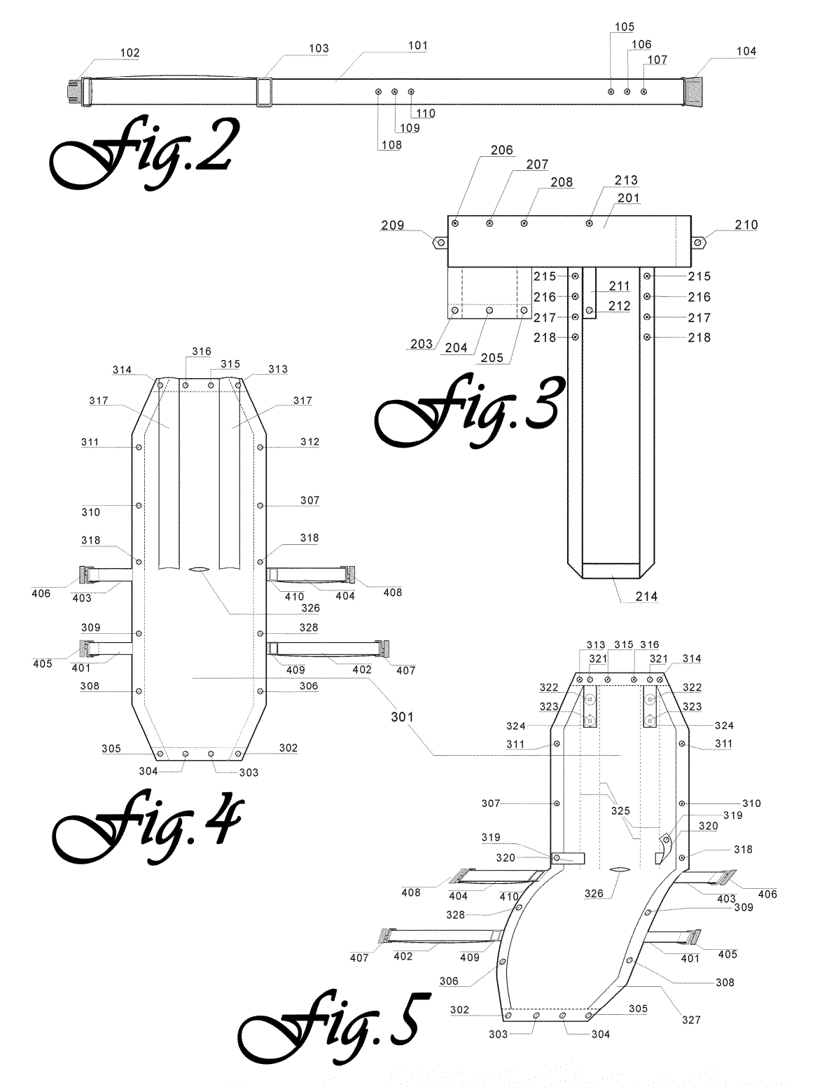

FIG. 2 illustrates the belt which is use to stabilize the invention to the wearer's waist. It comprises of 101, a fabric woven belt, belt buckle 102 and 104 installed at the opposite ends of the belt. A tri-glide button 103 is used so the wearer can adjust the belt in accordance to his/her measurement. Snap fasteners 105, 106 and 107 are used to attach to 209 on FIG. 3, as well as 108, 109 and 110 are used to attached to 210 on FIG. 3, wearer can use these sets of snap fasteners to adjust the enclosure piece which in shown on FIG. 3.

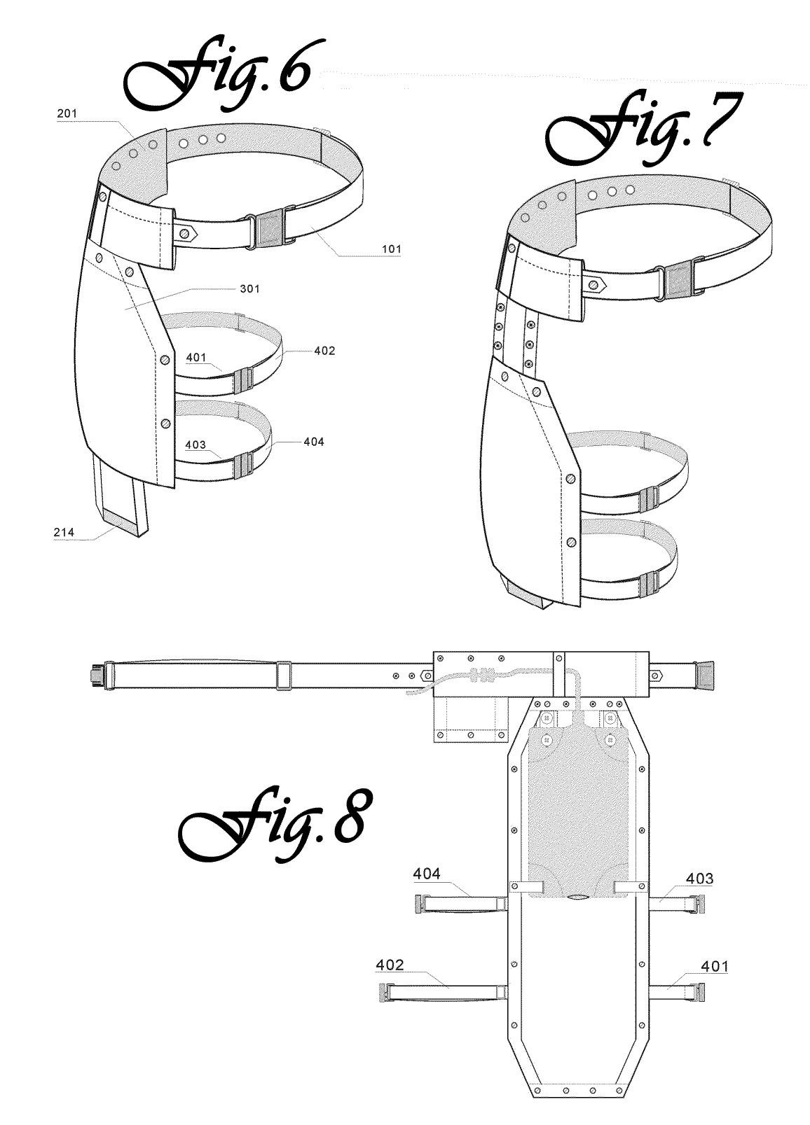

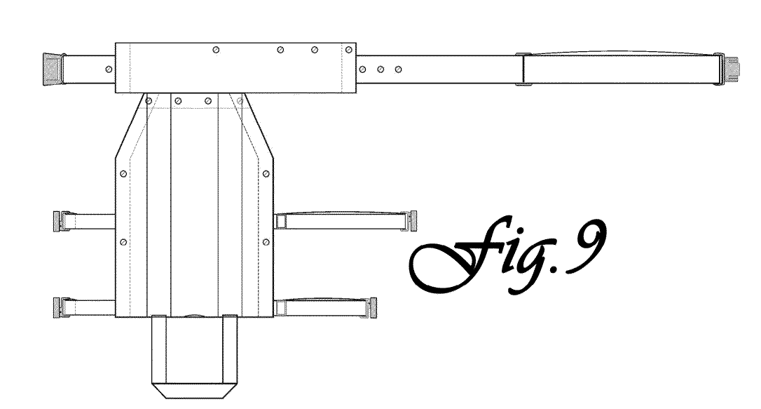

FIG. 3 illustrates the details of the adjustable enclosure that connects the belt and the pouch, it comprises of 201, a piece of fabric that creates an enclosure pathway where the belt can pass through. 203 and 206, 204 and 207, 205 and 208 are three sets of snap fasteners that create an enclosure that protects the valve of the drainage tube from the body. A small piece of rectangular fabric 211 is held upward by snaps 212 and 213. U-shape fabric woven tape 214 suspends from 210, together with snap sets 215, 216, 217 and 218, suspend and immobilize the pouch shown on FIG. 4 and FIG. 5.

FIG. 4 illustrates the detail exterior view of the pouch of the present Urinary Leg Bag Holder and Straps. Pouch is created by folding octagonal fabric with one set of long sides 301, connected at the boarders and stabilized by snap fasteners 308 and 311, 309 and 310, 306 and 312, 307 and 328, 305 and 314, 304 and 316, 303 and 315, 302 and 313. Two identical long rectangular fabric pieces 317 are sewed onto 301 by stitches 325 on FIG. 5 on the long sides create two pathways which would allow the U-shape tape 214 to pass through to suspend the pouch to the adjustable enclosure shown on FIG. 3. Opening 326 on the pouch would allow the drain valve to pass through, which the patient can easily drain the urine without having to take off the invention. The pouch is fasten to the wearer's thigh with four stretchable straps 401 and 402, 403 and 404, encircled the wearer's thigh and stabilizes with buckle 405 and 407, 406 and 408. Straps 402 and 404 are adjustable with two tri-glide button 409 and 410 respectively.

FIG. 5 illustrates the detail interior view of the pouch of the present Urinary Leg Bag Holder and Straps. Two small rectangular woven tapes 320 are sewn onto 301 on one short side and fasten on the other short side by two sets of snap fasteners 318 and 319. Snaps 321 are used to immobilize the pouch onto the U-shape tape 214, adjustable vertically by snapping 321 on 215, 216, 217 and 218. Two small pieces of rectangular tapes 324 are sew onto 301, providing support for two sets of buttons 322 and 323, along with 320, are used to stabilize the vinyl or plastic leg bag to the pouch.

The different components of this invention can vary in size in accordance of the wearer, as well as the position of the indwelling drainage tube. Natural materials are ideal for making the belt, the enclosure and pouch to mitigate irritation and allergic reaction of the wearer. The leg straps should be made of stretchable material to allow wearer more flexibility and comfort irrespective of position and movement. All materials used to make this Urinary Leg Bag Holder and Straps should be durable and washable.

1. The device comprises of:
A waist encircling belt with adjustable length;
An adjustable enclosure to the belt with a U shape fabric woven tape suspending from the said enclosure, comprising a piece of fabric that creates a pathway where the belt can pass through and the enclosure itself adjustable on the belt to the location of the drainage tube on the patient;
A pouch for holding a urine drainage bag, said pouch comprising an octagonal fabric folding upwards along its long sides to create a front and back panel with snap fasteners and the urine leg bag is held and adjustable by means of 2 sets of buttons;
Two sets of encircling straps extending from the middle and bottom portions of said pouch respectively, for stabilizing the pouch to the wearer's thigh, said straps are adjustable to the size of the wearer by means of two tri-glide button.

2. The device of claim 1 wherein the said enclosure is attached to the belt by snap fasteners at 3 different adjustable locations.

3. The device of claim 1 wherein the said pouch is connected to the said enclosure by the U-shape tape suspending from the said enclosure at 4 locations, where the pouch is adjustable vertically in accordance to the wearer's preference.

4. The device of claim 1 wherein the said encircling straps is attached to the front panel of the said pouch at two locations-middle and bottom portion.

5. The device of claim 4 wherein the said straps are made of stretchable material. The device of claim 1 wherein the belt, the enclosure and straps are all made of a woven material; fasteners are of stainless steel or plastic material; and belt buckles are of plastic or rubber material.
